(19) United States
(12) Patent Application Publication
(10) Pub. No.: US 2025/0117934 A1
(43) Pub. Date: Apr. 10, 2025

(54) MEDICAL INFORMATION PROCESSING APPARATUS, MEDICAL INFORMATION PROCESSING METHOD, AND COMPUTER-READABLE STORAGE MEDIUM

(71) Applicants: CANON MEDICAL SYSTEMS CORPORATION, Tochigi (JP); THE BRIGHAM AND WOMEN’S HOSPITAL, Inc., Boston, MA (US)

(72) Inventors: Yasuko FUJISAWA, Nasushiobara (JP); Mami TAKAHASHI, Nasushiobara (JP); Kei MORI, Brookline, MA (US); David R. WALT, Boston, MA (US)

(73) Assignees: CANON MEDICAL SYSTEMS CORPORATION, Tochigi (JP); THE BRIGHAM AND WOMEN’S HOSPITAL, Inc., Boston, MA (US)

(21) Appl. No.: 18/911,790

(22) Filed: Oct. 10, 2024

Related U.S. Application Data
(60) Provisional application No. 63/589,077, filed on Oct. 10, 2023.

(51) Int. Cl. G06T 7/00; G16H 30/20; G16H 30/40; G16H 50/20

(52) U.S. Cl. CPC G06T 7/0012; G16H 30/20; G16H 30/40; G16H 50/20; G06T 2207/30068; G06T 2207/30096

(57) ABSTRACT

A medical information processing apparatus according to an embodiment includes a first analysis unit, a second analysis unit, a determination unit, and a third analysis unit. The first analysis unit executes a first analysis based on a medical image of a subject. The second analysis unit executes a second analysis based on a specimen examination result of the subject. The determination unit determines a matching degree between the result of the first analysis by the first analysis unit and the result of the second analysis by the second analysis unit. The third analysis unit executes a third analysis based on the medical image and the specimen examination result according to analysis conditions based on the determination result of the matching degree by the determination unit, and determines diagnosis support information based on the medical image and the specimen examination result.

### CROSS REFERENCE TO RELATED APPLICATIONS

This application is based upon and claims the benefit of priority from the prior U.S. Provisional Patent Application No. 63/589,077, filed on Oct. 10, 2023, the entire contents of which are incorporated herein by reference.

### FIELD

Embodiments disclosed in the present specification and the drawings relate to a medical information processing apparatus, a medical information processing method, and a computer-readable storage medium.

### BACKGROUND

In the related art, image diagnosis for diagnosing cancer of a subject based on image information of the subject has been known. However, there is a case where it is difficult to diagnose cancer based on only image information since there is a case where images to be determined to be malignant cancer are different depending on cancer subtypes.

In addition, in the related art, a liquid biopsy that performs examination of cancer of a subject using a body fluid such as blood is known as an examination with a low burden on the subject. In recent years, a diagnostic technique in which examination information is added by a liquid biopsy to image information obtained by image diagnosis has been developed. However, the image information is morphological information, whereas the examination information by a liquid biopsy is property information. Therefore, the image information and the examination information a by liquid biopsy may not correspond to each other. Accordingly, in the related art, it has been difficult to improve lesion determination accuracy.

### BRIEF DESCRIPTION OF THE DRAWINGS

FIG. 1 is a block diagram illustrating a configuration example of a medical information processing system according to an embodiment;

FIG. 2 is a block diagram illustrating a configuration example of a medical information processing apparatus according to an embodiment;

FIG. 3 is a flowchart illustrating an operation example of a medical information processing apparatus according to an embodiment;

FIG. 4 is a view illustrating a calculation example of a matching degree in the operation example of the medical information processing apparatus according to an embodiment;

FIG. 5 is a flowchart illustrating a step of setting analysis conditions of a third analysis in the operation example of the medical information processing apparatus according to an embodiment;

FIG. 6 is a view illustrating a setting example of the analysis conditions of the third analysis in the operation example of the medical information processing apparatus according to an embodiment; and

FIG. 7 is a view illustrating a degree of contribution of attributes of a biomarker to a second analysis in the operation example of the medical information processing apparatus according to an embodiment.

### DETAILED DESCRIPTION

Hereinafter, embodiments of a medical information processing apparatus will be described with reference to the drawings. Note that, in the following description, components having substantially the same functions and configurations are denoted by the same reference numerals, and redundant description will be given only when necessary.

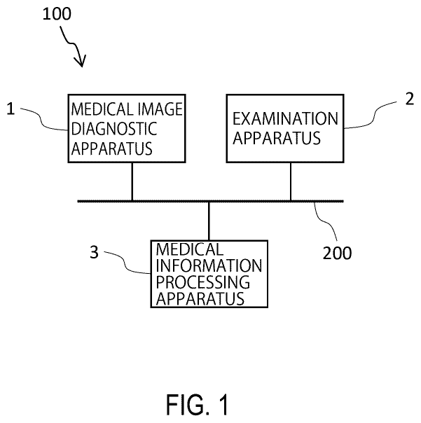

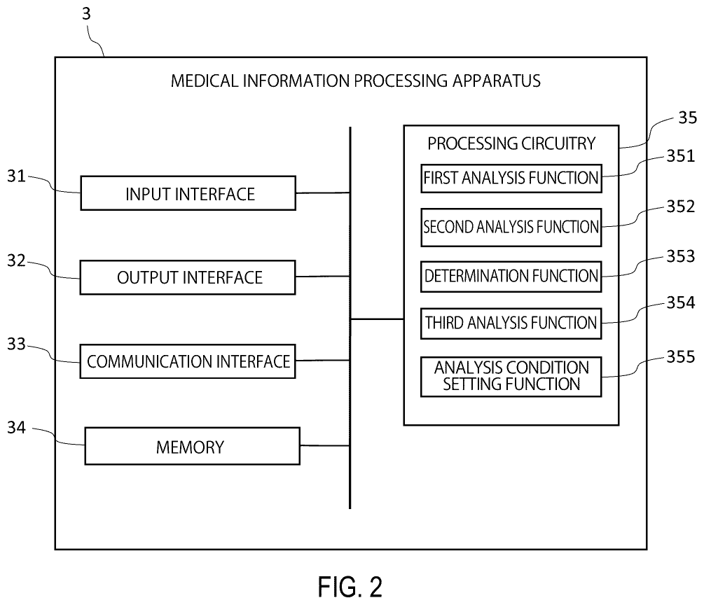

FIG. 1 is a block diagram illustrating a configuration example of a medical information processing system 100 according to an embodiment. FIG. 2 is a block diagram illustrating a configuration example of a medical information processing apparatus 3 according to a first embodiment.

As illustrated in FIG. 1, the medical information processing system 100 according to the first embodiment includes a medical image diagnostic apparatus 1, an examination apparatus 2, and a medical information processing apparatus 3. The medical image diagnostic apparatus 1, the examination apparatus 2, and the medical information processing apparatus 3 are communicably connected via a network 200. That is, the medical information processing apparatus 3 is provided on the medical information processing system 100 connected via the network 200. Note that the medical information processing apparatus 3 may be an offline apparatus. In this case, the medical information processing apparatus 3 may acquire information from the medical image diagnostic apparatus 1 and the examination apparatus 2 via a portable recording medium.

The respective apparatuses included in the medical information processing system 100 may be in a state of being able to communicate with each other directly or indirectly by, for example, an in-hospital local area network (LAN) installed in a hospital. In addition, an apparatus (for example, a server or the like that stores medical information) other than those illustrated in FIG. 1 may be communicably connected to the medical information processing system 100 illustrated in FIG. 1.

For example, the medical information processing system 100 may include various systems such as an electronic health record (EHR) system, a picture archiving and communication system (PACS), a hospital information system (HIS), a radiology information system (RIS), and a laboratory information system (LIS).

The medical image diagnostic apparatus 1 images a subject and collects a medical image. Then, the medical image diagnostic apparatus 1 transmits the collected medical image to the medical information processing apparatus 3. For example, the medical image diagnostic apparatus 1 may be an X-ray diagnostic apparatus such as a mammography apparatus, an X-ray computed tomography (CT) apparatus, a magnetic resonance imaging (MRI) apparatus, an ultrasonic diagnostic apparatus, a single photon emission computed tomography (SPECT) apparatus, a positron emission computed tomography (PET) apparatus, or the like.

The examination apparatus 2 is operated by a clinical laboratory technician or the like, and executes a specimen examination for analyzing a specimen such as blood or urine acquired from a patient. The specimen examination is, for example, a pathological examination, a blood/biochemical examination, a liquid biopsy examination, a general examination of urine, feces, or the like, an immune serum examination, a genetic examination, a microorganism examination, a blood transfusion or organ transplant-related examination, or the like. Then, the examination apparatus 2 transmits an examination result (measurement data or the like) of the specimen examination to the medical information processing apparatus 3.

For example, the examination apparatus 2 may be a biochemical automatic analysis apparatus, an immune automatic analysis apparatus, a flow cytometer, a gene analysis apparatus, a protein analysis apparatus, an extracellular vesicle analysis apparatus, a circulating tumor cell detection apparatus, or the like. Note that the flow cytometer is a device that performs flow cytometry. Flow cytometry is an analysis method in which a suspension of an object to be measured is used as a high-speed fluid, scattered light and fluorescence generated by irradiating the fluid with laser light, mercury light, or the like are measured, and the size, amount, or the like of the object to be measured is measured.

The gene analysis apparatus is an apparatus for analyzing a gene sequence. For example, the gene analysis apparatus is an apparatus that amplifies a nucleic acid molecule extracted from a biological sample by a polymerase chain reaction (PCR) method or the like and performs determination of the presence or absence of a specific gene sequence and quantitative determination, or an apparatus that analyzes sequence information of a nucleic acid molecule. The protein analysis apparatus is, for example, an automatic immunoassay analysis apparatus, a highly sensitive protein detection apparatus (for example, Single Molecule Assay Apparatus SIMOA (registered trademark), manufactured by Quanterix Inc.), or the like.

In addition, the extracellular vesicle analysis apparatus is, for example, a single extracellular vesicle analysis apparatus (for example, EXOVIEW (registered trademark), manufactured by NanoView Biosciences, Inc.), or the like. In addition, the circulating tumor cell detection apparatus is, for example, a circulating tumor cell detection apparatus (for example, CELLSEARCH (registered trademark) system, manufactured by Veridex, LLC), or the like.

The medical information processing apparatus 3 acquires various types of information from the medical image diagnostic apparatus 1 and the examination apparatus 2, and performs various types of information processing using the acquired information. For example, the medical information processing apparatus 3 is realized by computer equipment such as a server, a workstation, a personal computer, or a tablet terminal.

As illustrated in FIG. 2, the medical information processing apparatus 3 includes an input interface 31, an output interface 32, a communication interface 33, a memory 34, and a processing circuit 35. The input interface 31, the output interface 32, the communication interface 33, and the memory 34 are connected to the processing circuit 35.

The input interface 31 receives various instructions and information input operations from an operator. Specifically, the input interface 31 converts an input operation received from the operator into an electrical signal and outputs the electrical signal to the processing circuit 35. For example, the input interface 31 is realized by a trackball, a switch button, a mouse, a keyboard, a touch pad that performs an input operation by touching an operation surface, a touch screen in which a display screen and a touch pad are integrated, a non-contact input circuit using an optical sensor, a sound input circuit, or the like. Note that the input interface 31 is not limited to one including physical operation components such as a mouse and a keyboard. For example, an electric signal processing circuit that receives an electric signal corresponding to an input operation from an external input device provided separately from the apparatus and outputs the electric signal to a control circuit is also included in the example of the input interface 31.

The output interface 32 outputs various types of information. For example, the output interface 32 includes a display. The display outputs a graphical user interface (GUI) or the like for receiving the medical image generated by the processing circuit 35 and various operations from the operator. For example, a liquid crystal display (LCD), a cathode ray tube (CRT) display, an organic electroluminescence display (OELD), a plasma display, or any other display can be appropriately used as the display. The output interface 32 may include a speaker.

The communication interface 33 controls communication performed with each apparatus in the medical information processing system 100. Specifically, the communication interface 33 receives various types of information from each apparatus, and outputs the received information to the processing circuit 35. For example, the communication interface 33 is realized by a network card, a network adapter, a network interface controller (NIC), or the like.

The memory 34 is a non-transitory storage device that stores various types of information, and is, for example, a hard disk drive (HDD), an optical disk, a solid state drive (SSD), an integrated circuit storage device, or the like. The memory 34 stores, for example, a control program for controlling the medical information processing apparatus 3 and various types of data used for executing the control program. The memory 34 may be a drive device that reads and writes various types of information from and to a portable storage medium such as a compact disc (CD), a digital versatile disc (DVD), or a flash memory, a semiconductor memory element such as a random access memory (RAM), or the like, in addition to the HDD, the SSD, and the like.

The processing circuit 35 is a circuit that controls the entire operation of the medical information processing apparatus 3 according to an electric signal of an input operation input from the input interface 31. For example, the processing circuit 35 includes a first analysis function 351, a second analysis function 352, a determination function 353, a third analysis function 354, and an analysis condition setting function 355.

Here, for example, each processing function executed by each of the first analysis function 351, the second analysis function 352, the determination function 353, the third analysis function 354, and the analysis condition setting function 355, which are the components of the processing circuit 35 illustrated in FIG. 2, is recorded in the memory 34 in the form of a computer-executable program. The processing circuitry 35 is, for example, a processor. The processor constituting the processing circuitry 35 reads each program from the memory 34 and executes the program to realize a function corresponding to each read program. In other words, the processing circuitry 35 in a state of reading each program has each function illustrated in the processing circuitry 35 of FIG. 2.

Note that, in FIG. 2, although a case where a processing function of each of the first analysis function 351, the second analysis function 352, the determination function 353, the third analysis function 354, and the analysis condition setting function 355 is realized by a single processing circuitry 35 is described, an embodiment is not limited thereto. For example, the processing circuitry 35 may be configured by combining a plurality of independent processors, and each processor may execute each program to realize each processing function. In addition, each processing function of the processing circuitry 35 may be realized by being appropriately distributed or integrated into a single or a plurality of processing circuits.

The first analysis function 351 executes a first analysis based on a medical image of a subject. The first analysis function 351 may determine the presence or absence of a tumor in a medical image in the first analysis. In addition, in a case where it is determined in the first analysis that a tumor is present, the first analysis function 351 may determine whether the tumor is benign or malignant. The first analysis may be performed by setting a region of interest (ROI) on a medical image suspected of the presence of a tumor. The first analysis may be performed using machine learning ML. The first analysis may be performed using a learned model obtained by learning the tumor on the medical image. The machine learning ML may be deep learning DL using a neural network. In a case where the information indicating the region of interest set by the medical image diagnostic apparatus 1 is acquired by the medical information processing apparatus 3, the first analysis function 351 may perform the first analysis based on information indicating the region of interest. The first analysis function 351 may calculate the result of the first analysis together with likelihood thereof.

The first analysis function 351 may execute the first analysis further based on the past medical information of the subject. For example, the first analysis function 351 may acquire the past medical information of the subject from an electronic health record or a personal health record (PHR) and may use the information for the first analysis.

The second analysis function 352 executes a second analysis based on a specimen examination result of the subject. The specimen examination result may include a value of a biomarker contained in a specimen (for example, a body fluid such as blood or urine) collected from a subject. The second analysis function 352 may execute the second analysis based on the value of the biomarker. That is, the second analysis function 352 may execute the second analysis based on a result of a liquid biopsy related to the subject. In the liquid biopsy, analysis results of genes (DNA, RNA, mRNA, cfDNA, and the like), proteins, circulating cancer cells (CTCs), and the like are obtained. The second analysis function 352 may determine the presence or absence of a lesion based on whether or not a value of a biomarker indicating a lesion is detected in the second analysis. The lesion is, for example, a cancer such as breast cancer. The second analysis function 352 may execute the second analysis using the machine learning ML. The second analysis function 352 may execute the second analysis using the deep learning DL as the machine learning ML. The second analysis function 352 may calculate the result of the second analysis together with likelihood thereof. In addition, in the second analysis using machine learning, a degree of contribution in which the value of each biomarker contributes to the result of the analysis may be calculated together with the result of the analysis.

The second analysis function 352 may execute the second analysis further based on the past medical information of the subject. For example, the second analysis function 352 may acquire the past medical information of the subject from an electronic health record or a personal health record (PHR) and may use the information for the second analysis.

The determination function 353 determines a matching degree between the result of the first analysis by the first analysis function 351 and the result of the second analysis by the second analysis function 352. The determination function 353 may determine the matching degree in consideration of the likelihood of the result of the first analysis and the likelihood of the result of the second analysis.

The third analysis function 354 executes the third analysis based on the medical image and the specimen examination result according to the analysis conditions based on the determination result of the matching degree by the determination function 353. The third analysis function 354 executes the third analysis and determines diagnosis support information based on the medical image and the specimen examination result. The diagnosis support information may be information indicating whether the tumor is benign or malignant. The third analysis function 354 executes the third analysis using machine learning, that is, a learning algorithm.

The analysis condition setting function 355 sets analysis conditions of the third analysis by setting at least one of a parameter (that is, the parameter of the algorithm) of machine learning used for the third analysis, a mode, and a network. The third analysis function 354 executes the third analysis according to the analysis condition set by the analysis condition setting function 355. The parameter of the machine learning used for the third analysis may include a weight of an attribute of the biomarker contributing to the third analysis using the machine learning.

The analysis condition setting function 355 may determine the purpose of the third analysis, that is, the algorithm used for the third analysis, based on the determination result of the matching degree by the determination function 353, the result of the first analysis by the first analysis function 351, and the result of the second analysis by the second analysis function 352. That is, the analysis condition setting function 355 may determine the purpose of the third analysis based on the determination result of the matching degree by the determination function 353 and the result of the comprehensive determination of the lesion based on the result of the first analysis and the result of the second analysis. Further, the analysis condition setting function 355 may determine the attributes of the biomarkers to be noted based on the determined purpose of the third analysis, that is, the algorithm. Then, the analysis condition setting function 355 may set the weight of the attribute of the biomarker contributing to the third analysis by performing weighting on the determined attribute according to the degree of contribution of the determined attribute contributing to the second analysis.

The attributes of the biomarkers of interest may be the use of biomarkers. For example, the use of the biomarker may be (1) a general biomarker for diagnosis and treatment evaluation of cancer, (2) determination of subtype, (3) differentiation from normal cell types such as lumen, base, and myoepithelium, (4) discrimination of proliferation and disease progression state, (5) a biomarker associated with prognosis in cancer immunohistochemistry, and the like.

In addition, in a case where the biomarker is applied to breast cancer, the attribute of the biomarker to be noted (that is, the use) may be (1) a general-purpose tumor marker (that is, a tumor marker common to cancer in general), (2) determination of triple negative breast cancer, (3) determination of a subtype other than triple negative breast cancer, (4) detection of metastasis, (5) early detection, (6) determination of fibroadenoma such as cyst, (7) determination of cancer recurrence and the like.

The analysis condition setting function 355 may determine the attribute of the biomarker to be noted based on a correspondence relationship between the type of biomarker (biomarker used in the second analysis) stored in advance in the memory 34 and the attribute of the biomarker. In addition, the analysis condition setting function 355 may acquire a correspondence relationship between the type of the biomarker used in the second analysis and the attribute thereof based on information held in an external database, academic institute report information on an external network, research information stored in the operator's own computer, and the like, and may determine the attribute of the biomarker to be noted based on the acquired correspondence relationship.

When the degree of contribution of the determined attribute of the biomarker to be noted contributing to the second analysis is low, the analysis condition setting function 355 may weight to the attribute of the determined biomarker to be noted more heavily than when the degree of contribution is high.

From the result of the machine learning used for the second analysis, the analysis condition setting function 355 may acquire the degree of contribution of the attribute of the biomarker to be noted contributing to the second analysis. Furthermore, the analysis condition setting function 355 may calculate a score according to the acquired degree of contribution. The score when the degree of contribution is high may be a value larger than the score when the degree of contribution is low. Then, the analysis condition setting function 355 may perform weighting on the attribute of the determined biomarker to be noted based on the calculated score.

The analysis condition setting function 355 may set analysis conditions (that is, parameters, modes, networks, and the like) for reducing the false negative determination in a case where the matching degree as to whether the tumor is benign or malignant is greater than a first threshold and the first and second analysis results are benign.

In addition, in a case where the matching degree is greater than the first threshold and the first and second analysis results are malignant, the analysis condition setting function 355 may set analysis conditions for reducing the false positive determination or detecting metastasis and recurrence of a lesion.

In addition, in a case where the matching degree is equal to or less than the first threshold and is equal to or greater than a second threshold, which is less than the first threshold, the analysis condition setting function 355 may set analysis conditions for reducing the false negative determination or detecting metastasis and recurrence of a lesion.

In addition, in a case where the matching degree is less than the second threshold and the result of the first analysis is benign or absent, the analysis condition setting function 355 may set analysis conditions for early detection of a lesion.

In addition, in a case where the matching degree is less than the second threshold and the result of the first analysis is benign, the analysis condition setting function 355 may set analysis conditions for reducing the false positive determination based on an image or detecting metastasis and recurrence of a lesion.

In addition, in a case where the matching degree is less than the second threshold and the result of the first analysis is malignant, the analysis condition setting function 355 may set analysis conditions for reducing the false negative determination based on an image or detecting metastasis and recurrence of a lesion.

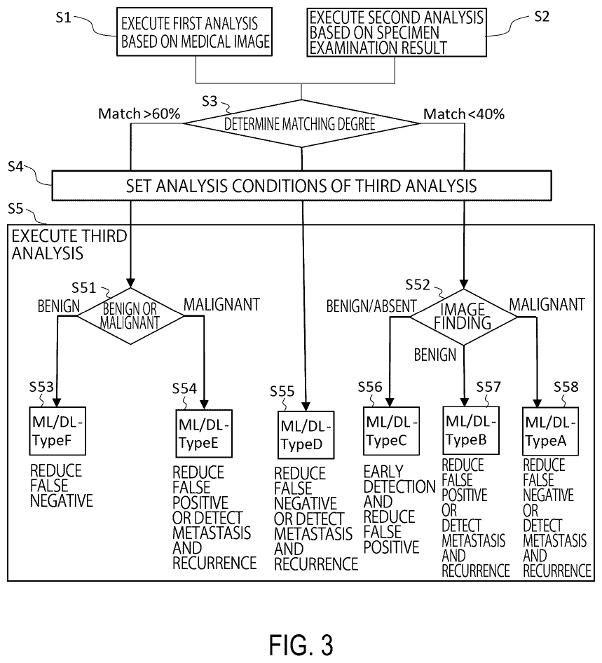

Next, an operation example of the medical information processing apparatus 3 according to the first embodiment configured as described above will be described. FIG. 3 is a flowchart illustrating an operation example of the medical information processing apparatus 3 according to the first embodiment. Note that, in the following operation example, it is assumed that the specimen examination is a liquid biopsy (LB).

First, as illustrated in FIG. 3, the first analysis function 351 executes the first analysis based on a medical image (Step S1).

In addition, the second analysis function 352 executes the second analysis based on a specimen examination result of the subject (Step S2).

Next, the determination function 353 determines a matching degree between the result of the first analysis by the first analysis function 351 and the result of the second analysis by the second analysis function 352 (Step S3). In addition, the determination function 353 performs comprehensive determination based on the result of the first analysis by the first analysis function 351 and the result of the second analysis by the second analysis function 352.

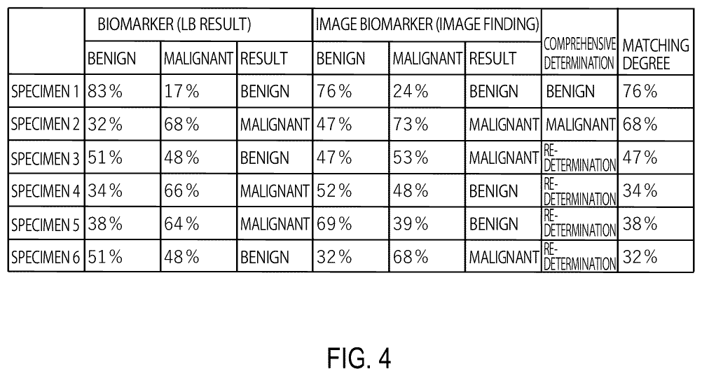

FIG. 4 is a view illustrating a calculation example of a matching degree in the operation example of the medical information processing apparatus 3 according to an embodiment. For example, in a case where the subject is a “specimen 1” in FIG. 4, the determination function 353 determines that the matching degree is 76% since the result of the second analysis (LB result) is benign (likelihood of benign: 83%, likelihood of malignant: 14%) and the result of the first analysis (image findings) is benign (likelihood of benign: 76%, likelihood of malignant: 24%). The matching degree corresponds to the benign likelihood in the result of the first analysis with the smaller benign likelihood. In this case, as illustrated in FIG. 4, the determination function 353 determines that the lesion of the “specimen 1” is benign as the comprehensive determination based on the result of the first analysis and the result of the second analysis.

In addition, in a case where the subject is a “specimen 2” in FIG. 4, the determination function 353 determines that the matching degree is 68% since the result of the second analysis is malignant (likelihood of benign: 32%, likelihood of malignant: 68%) and the result of the first analysis is malignant (likelihood of benign: 47%, likelihood of malignant: 73%). The matching degree corresponds to the malignant likelihood in the result of the second analysis with the smaller malignant likelihood. In this case, as illustrated in FIG. 4, the determination function 353 determines that the lesion of the “specimen 2” is malignant as the comprehensive determination based on the result of the first analysis and the result of the second analysis.

In addition, in a case where the subject is a “specimen 3” in FIG. 4, the determination function 353 determines that the matching degree is 47% since the result of the second analysis is benign (likelihood of benign: 51%, likelihood of malignant: 48%) and the result of the first analysis is malignant (likelihood of benign: 47%, likelihood of malignant: 53%). The matching degree corresponds to the benign likelihood in the result of the first analysis with the smaller benign likelihood. In the example illustrated in FIG. 4, in a case where the result of the second analysis and the result of the first analysis are different in whether they are benign or malignant as in, for example, the “specimen 3”, the determination function 353 determines, as the matching degree, the smallest likelihood among the four likelihoods of the benign and malignant likelihoods of the result of the second analysis and the benign and malignant likelihoods of the result of the first analysis. The same applies to a “specimen 4” to a “specimen 6” described below. In this case, as illustrated in FIG. 4, the result of the comprehensive determination by the determination function 353 for the “specimen 3” requires re-determination.

In addition, in a case where the subject is the “specimen 4” in FIG. 4, the determination function 353 determines that the matching degree is 34% since the result of the second analysis is malignant (likelihood of benign: 34%, likelihood of malignant: 66%) and the result of the first analysis is benign (likelihood of benign: 52%, likelihood of malignant: 48%). The matching degree corresponds to the benign likelihood in the result of the second analysis with the smaller benign likelihood. In this case, as illustrated in FIG. 4, the result of the comprehensive determination by the determination function 353 for the “specimen 4” requires re-determination.

In addition, in a case where the subject is the “specimen 5” in FIG. 4, the determination function 353 determines that the matching degree is 38% since the result of the second analysis is malignant (likelihood of benign: 38%, likelihood of malignant: 64%) and the result of the first analysis is benign (likelihood of benign: 69%, likelihood of malignant: 39%). The matching degree corresponds to the benign likelihood in the result of the second analysis with the smaller benign likelihood. In this case, as illustrated in FIG. 4, the result of the comprehensive determination by the determination function 353 for the “specimen 5” requires re-determination.

In addition, in a case where the subject is the “specimen 6” in FIG. 4, the determination function 353 determines that the matching degree is 32% since the result of the second analysis is benign (likelihood of benign: 51%, likelihood of malignant: 48%) and the result of the first analysis is malignant (likelihood of benign: 32%, likelihood of malignant: 68%). The matching degree corresponds to the benign likelihood in the result of the first analysis with the smaller benign likelihood. In this case, as illustrated in FIG. 4, the result of the comprehensive determination by the determination function 353 for the “specimen 6” requires re-determination.

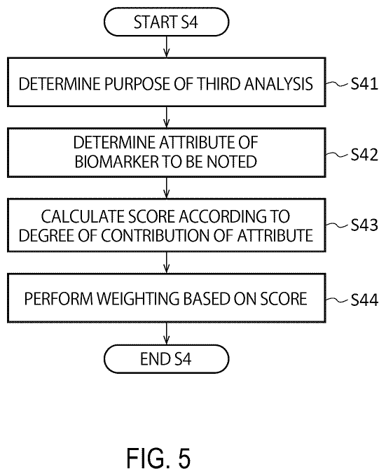

After the determination of the matching degree and the comprehensive determination are performed, as illustrated in FIG. 3, the analysis condition setting function 355 sets analysis conditions of the third analysis (Step S4). FIG. 5 is a flowchart illustrating a step of setting the analysis conditions of the third analysis in the operation example of the medical information processing apparatus 3 according to an embodiment.

Specifically, as illustrated in FIG. 5, first, the analysis condition setting function 355 determines the purpose of the third analysis, that is, an algorithm used for the third analysis, based on the determination result of the matching degree and the result of the comprehensive determination (Step S41).

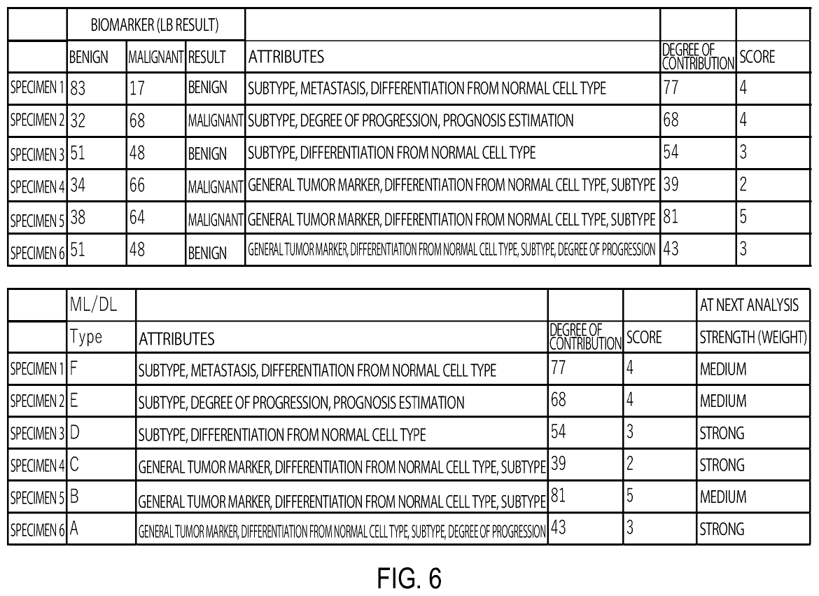

FIG. 6 is a view illustrating a setting example of the analysis conditions of the third analysis in the operation example of the medical information processing apparatus according to an embodiment. In the examples illustrated in FIGS. 4 and 6, in a case where the subject is the “specimen 1”, the analysis condition setting function 355 determines that the purpose of the third analysis, that is, the algorithm is to reduce false negative (“ML/DL-Type F”) based on the matching degree of 76%, which is larger than 60% (that is, the first threshold value), and the result of the comprehensive determination of benign.

In addition, in a case where the subject is the “specimen 2”, the analysis condition setting function 355 determines that the purpose of the third analysis, that is, the algorithm is to reduce false positive or to detect metastasis and recurrence (“ML/DL-Type E”) based on the matching degree of 68%, which is larger than 60%, and the result of the comprehensive determination of malignant.

In addition, in a case where the subject is the “specimen 3”, the analysis condition setting function 355 determines that the purpose of the third analysis, that is, the algorithm is to reduce false negative on the image or to detect metastasis and recurrence (“ML/DL-Type D”) based on the matching degree of 40% (that is, the second threshold) or more and 47%, which is less than 60%, and the result of the comprehensive determination of re-determination.

In addition, in a case where the subject is the “specimen 4”, the analysis condition setting function 355 determines that the purpose of the third analysis, that is, the algorithm is to reduce false positive on early detection and image (“ML/DL-Type C”) based on the matching degree of 34%, which is less than 40%, and the result of the comprehensive determination of re-determination.

In addition, in a case where the subject is the “specimen 5”, the analysis condition setting function 355 determines that the purpose of the third analysis, that is, the algorithm is to reduce false positive on the image or to detect metastasis and recurrence (“ML/DL-Type B”) based on the matching degree of 38%, which is less than 40%, and the result of the comprehensive determination of re-determination.

In addition, in a case where the subject is the “specimen 6”, the analysis condition setting function 355 determines that the purpose of the third analysis, that is, the algorithm is to reduce false negative on the image or to detect metastasis and recurrence (“ML/DL-Type A”) based on the matching degree of 32%, which is less than 40%, and the result of the comprehensive determination of re-determination.

After determining the purpose of the third analysis, as illustrated in FIG. 5, the analysis condition setting function 355 determines the attributes of the biomarker to be noted based on the determined purpose of the third analysis (Step S42). In the example illustrated in FIGS. 4 and 6, in a case where the subject is the “specimen 1”, the analysis condition setting function 355 determines that the attributes of the biomarker to be noted are determination of subtype, metastasis determination, and differentiation from a normal cell type. In addition, in a case where the subject is the “specimen 2”, the analysis condition setting function 355 determines that the attributes of the biomarker to be noted are determination of subtype, determination of degree of progression, and prognosis estimation. In addition, in a case where the subject is the “specimen 3”, the analysis condition setting function 355 determines that the attributes of the biomarker to be noted are determination of subtype and differentiation from a normal cell type. In addition, in a case where the subject is the “specimen 4”, the analysis condition setting function 355 determines that the attributes of the biomarker to be noted are a general tumor disease marker, differentiation from a normal cell type, and determination of subtype. In addition, in a case where the subject is the “specimen 5”, the analysis condition setting function 355 determines that the attributes of the biomarker to be noted are a general tumor disease marker, differentiation from a normal cell type, and determination of subtype. In addition, in a case where the subject is the “specimen 6”, the analysis condition setting function 355 determines that the attributes of the biomarker to be noted are a general tumor disease marker, differentiation from a normal cell type, determination of subtype, and prognosis estimation.

After determining the attributes of the biomarker to be noted, as illustrated in FIG. 5, the analysis condition setting function 355 calculates a score according to the degree of contribution of the determined attributes of the biomarker to be noted contributing to the second analysis (Step S43).

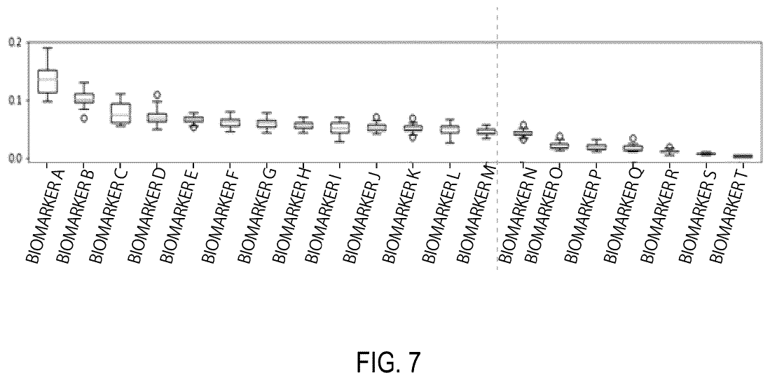

FIG. 7 is a view illustrating a degree of contribution of attributes of a biomarker to the second analysis in the operation example of the medical information processing apparatus 3 according to an embodiment. The vertical axis in FIG. 7 indicates the degree of contribution of each of attributes of each of the biomarkers A to T to the second analysis. The degree of contribution of each of the attributes of each of the biomarkers A to T varies as illustrated in FIG. 7. For example, the analysis condition setting function 355 calculates a score corresponding to a value obtained by summing degrees of contribution to the top 60% tiles for each attribute (for each of the biomarkers A to T). For example, the analysis condition setting function 355 may calculate a score “5” in a case where the degree of contribution (that is, the total value) is 80 to 100%. In addition, the analysis condition setting function 355 may calculate a score “4” in a case where the degree of contribution is 79 to 60%. In addition, the analysis condition setting function 355 may calculate a score “3” in a case where the degree of contribution is 59 to 40%. In addition, the analysis condition setting function 355 may calculate a score “2” in a case where the degree of contribution is 39 to 20%. In addition, the analysis condition setting function 355 may calculate a score “1” in a case where the degree of contribution is 19 to 0%.

In the examples illustrated in FIGS. 4 and 6, in a case where the subject is the “specimen 1”, the analysis condition setting function 355 calculates the score “4” based on the fact that the degree of contribution of the determined attributes of the biomarker to be noted contributing to the second analysis is 77%. In addition, in a case where the subject is the “specimen 2”, the analysis condition setting function 355 calculates the score “4” based on the fact that the degree of contribution of the determined attributes of the biomarker to be noted contributing to the second analysis is 68%. In addition, in a case where the subject is the “specimen 3”, the analysis condition setting function 355 calculates the score “3” based on the fact that the degree of contribution of the determined attributes of the biomarker to be noted contributing to the second analysis is 54%. In addition, in a case where the subject is the “specimen 4”, the analysis condition setting function 355 calculates the score “2” based on the fact that the degree of contribution of the determined attributes of the biomarker to be noted contributing to the second analysis is 39%. In addition, in a case where the subject is the “specimen 5”, the analysis condition setting function 355 calculates the score “5” based on the fact that the degree of contribution of the determined attributes of the biomarker to be noted contributing to the second analysis is 81%. In addition, in a case where the subject is the “specimen 6”, the analysis condition setting function 355 calculates the score “3” based on the fact that the degree of contribution of the determined attributes of the biomarker to be noted contributing to the second analysis is 43%.

After calculating the score, as illustrated in FIG. 5, the analysis condition setting function 355 performs weighting based on the calculated score to set a weight by which the determined attributes of the biomarker to be noted contributing to the third analysis (Step S44).

In the example illustrated in FIG. 6, in a case where the subject is the “specimen 1”, the analysis condition setting function 355 sets the weighting strength for the attributes “determination of subtype, determination of metastasis, and differentiation from a normal cell type” to “medium” for the third analysis according to the algorithm “ML/DL-Type F” for reducing false negative. Such weighting is based on the fact that the score of the “specimen 1” is the second highest value “4”.

In addition, in a case where the subject is the “specimen 2”, the analysis condition setting function 355 sets the weighting strength for the attributes “determination of subtype, determination of degree of progression, and prognosis estimation” to “medium” for the third analysis according to the algorithm “ML/DL-Type E” for reducing false positive or detecting metastasis and recurrence. Such weighting is based on the fact that the score of the “specimen 2” is the second highest value “4”. Note that the analysis condition setting function 355 may perform weighting to increase the weight of a biomarker for determining a subtype of breast cancer (subtyping) or the weight of a tumor biomarker of a site other than the breast (for example, prostate or the like) for the “specimen 2”.

In addition, in a case where the subject is the “specimen 3”, the analysis condition setting function 355 sets the weighting strength for the attributes “determination of subtype and differentiation from a normal cell type” to “strong” for the third analysis according to the algorithm “ML/DL-Type D” for reducing false negative on an image or detecting metastasis and recurrence. Such weighting is based on the fact that the score of the “specimen 3” is the medium value “3”.

In addition, in a case where the subject is the “specimen 4”, the analysis condition setting function 355 sets the weighting strength for the attributes “a general tumor disease marker, differentiation from a normal cell type, and determination of subtype” to “strong” for the third analysis according to the algorithm “ML/DL-Type C” for reducing false positive on early detection and an image. Such weighting is based on the fact that the score of the “specimen 4” is the second lowest value “2”.

For example, triple negative breast cancer generally has clear borders and appears to be nearly benign on images. Therefore, the analysis condition setting function 355 can reduce oversight of triple negative breast cancer and lead to early detection by performing weighting to increase the weight (degree of contribution) of many triple negative biomarkers for the “specimen 4” for which the analysis result of the first analysis is benign. Biomarkers common in triple negative breast cancer are, for example, an epidermal growth factor receptor (EGFR), an estrogen receptor (ER), a progesterone receptor (PR), and HER2/neu.

Note that, also in a case where the matching degree is less than 40% and there is no analysis result of the first analysis (that is, in a case where the cancer is not grown enough to be determined from the image), the analysis condition setting function 355 may perform weighting for the third analysis according to the algorithm “ML/DL-Type C” for early detection. In this case, the analysis condition setting function 355 may perform weighting to increase the weight of a biomarker having characteristics of early detection of cancer. Biomarkers having characteristics of early detection of cancer are, for example, DNA methylation analysis, tumor associated auto-antibody analysis, and the like.

In addition, in a case where the subject is the “specimen 5”, the analysis condition setting function 355 sets the weighting strength for the attributes “biomarker attributes are a general tumor disease marker, differentiation from a normal cell type, and determination of subtype” to “medium” for the third analysis according to the algorithm “ML/DL-Type B” for reducing false positive on an image or detecting metastasis and recurrence. Such weighting is based on the fact that the score of the “specimen 5” is the highest value “5”. For example, the analysis condition setting function 355 may perform weighting for the “specimen 5” to increase the weight of fibroblasts and tumor biomarkers.

In addition, in a case where the subject is the “specimen 6”, the analysis condition setting function 355 sets the weighting strength for the attributes “a general tumor disease marker, differentiation from a normal cell type, determination of subtype, and determination of degree of progression” to “strong” for the third analysis according to the algorithm “ML/DL-Type A” for reducing false negative on an image or detecting metastasis and recurrence. Such weighting is based on the fact that the score of the “specimen 6” is the medium value “3”. For example, the analysis condition setting function 355 may perform weighting to increase the weight of a biomarker for breast cancer subtyping or the weight of a tumor biomarker of a site other than the breast (for example, prostate or the like) for the “specimen 6”.

In a case where the weight of the biomarker for breast cancer subtyping increases, the analysis condition setting function 355 may increase, for a subtype corresponding to a feature of an image acquired by the first analysis, the weight of the biomarker associated with the subtype. For example, in a case where the feature of the image is that the boundary of the image is clear without calcification, the analysis condition setting function 355 may increase a weight of an epidermal growth factor receptor (positive), an estrogen receptor (negative), a progesterone receptor (negative), and HER2/neu (negative) associated with triple negative breast cancer. In addition, in a case where the feature of the image is that the image is accompanied by calcification, the analysis condition setting function 355 may increase the weight of HER2 (positive) associated with HER2-positive breast cancer. In addition, in a case where the feature of the image is that the boundary of the image is unclear or accompanied by calcification, the analysis condition setting function 355 may increase a weight of an estrogen receptor (positive), a progesterone receptor (positive), and HER2/neu (positive/negative) associated with luminal breast cancer.

After the analysis conditions of the third analysis are set as described above, as illustrated in FIG. 3, the third analysis function 354 executes the third analysis according to the set analysis conditions (Step S5).

For example, in a case where the subject is the “specimen 1” of FIGS. 4 and 6, that is, in a case where the first and second analysis results are benign (Step S51: benign), the third analysis function 354 executes the third analysis according to the analysis conditions (ML/DL-Type F) for reducing false negative (Step S53).

In addition, in a case where the subject is the “specimen 2” of FIGS. 4 and 6, that is, in a case where the first and second analysis results are malignant (Step S51: malignant), the third analysis function 354 executes the third analysis according to the analysis conditions (ML/DL-Type F) for reducing false positive or detecting metastasis and recurrence (Step S54).

In addition, in a case where the subject is the “specimen 3” of FIGS. 4 and 6, the third analysis function 354 executes the third analysis according to the analysis conditions (ML/DL-Type D) for reducing false negative on an image or detecting metastasis and recurrence (Step S55).

In addition, in a case where the subject is the “specimen 4” of FIGS. 4 and 6, that is, in a case where the result of the first analysis is benign or absent (Step S52: benign/absent), the third analysis function 354 executes the third analysis according to the analysis conditions (ML/DL-Type C) for reducing false positive on early detection (Step S56).

In addition, in a case where the subject is the “specimen 5” of FIGS. 4 and 6, that is, in a case where the result of the first analysis is benign (Step S52: benign), the third analysis function 354 executes the third analysis according to the analysis conditions (ML/DL-Type B) for reducing false positive on an image or detecting metastasis and recurrence (Step S57).

In addition, in a case where the subject is the “specimen 6” of FIGS. 4 and 6, that is, in a case where the result of the first analysis is malignant (Step S52: malignant), the third analysis function 354 executes the third analysis according to the analysis conditions (ML/DL-Type A) for reducing false negative on an image or detecting metastasis and recurrence (Step S58).

As described above, in an embodiment, the first analysis function 351 executes the first analysis based on a medical image of a subject. In addition, the second analysis function 352 executes the second analysis based on a specimen examination result of the subject. In addition, the determination function 353 determines a matching degree between the result of the first analysis by the first analysis function 351 and the result of the second analysis by the second analysis function 352. The third analysis function 354 executes the third analysis based on the medical image and the specimen examination result according to analysis conditions based on the determination result of the matching degree by the determination function 353, and determines diagnosis support information based on the medical image and the specimen examination result.

As a result, the third analysis is performed based on the analysis conditions based on the determination result of the matching degree between the result of the first analysis based on the medical image of the subject and the result of the second analysis based on the specimen examination result of the subject, such that appropriate diagnosis support information can be determined. Therefore, according to the first embodiment, the lesion determination accuracy can be improved.

In addition, in an embodiment, the specimen examination result includes a value of a biomarker contained in the specimen collected from the subject. In addition, the second analysis function 352 executes the second analysis based on the value of the biomarker. In addition, the third analysis function 354 executes the third analysis using the machine learning. In addition, the analysis condition setting function 355 sets analysis conditions by setting at least one of a parameter of machine learning, a mode, and a network. In addition, the third analysis function 354 executes the third analysis according to the analysis condition set by the analysis condition setting function 355.

As a result, since the third analysis can be performed with high accuracy, the lesion determination accuracy can be appropriately improved.

In addition, in an embodiment, the parameter is the weight of the attribute of the biomarker that contributes to the third analysis using machine learning.

As a result, the third analysis can be appropriately performed by setting the weight of the attribute of the biomarker in consideration of the determination result of the matching degree.

In addition, in an embodiment, the analysis condition setting function 355 may determine the purpose of the third analysis, based on the determination result of the matching degree by the determination function 353, the result of the first analysis by the first analysis function 351, and the result of the second analysis by the second analysis function 352. In addition, the analysis condition setting function 355 determines the attributes of the biomarkers of to be noted based on the determined purpose of the third analysis. In addition, the analysis condition setting function 355 sets the weight of the attribute of the biomarker contributing to the third analysis by performing weighting on the determined attribute according to the degree of contribution of the determined attribute contributing to the second analysis.

As a result, even in a case where the attribute of the biomarker to be noted is a minority (that is, the case where the degree of contribution of the attribute contributing to the second analysis is low), it is possible to set the analysis conditions of the third analysis in consideration of the attribute of the minority. Therefore, according to the embodiment, in a case where even a minority attribute is an attribute to be noted, the attribute can be appropriately reflected in the result of the third analysis.

In addition, in an embodiment, when the degree of contribution of the determined attribute contributing to the second analysis is low, the analysis condition setting function 355 weights the determined attribute more heavily than when the degree of contribution is high.

As a result, it is possible to more appropriately set the analysis conditions of the third analysis in consideration of the minority attribute.

In addition, in an embodiment, the second analysis function 352 executes the second analysis using machine learning. In addition, the analysis condition setting function 355 acquires the degree of contribution from the result of the machine learning used for the second analysis, calculates a score according to the acquired degree of contribution, and performs weighting on the determined attribute based on the calculated score.

As a result, appropriate weighting can be performed based on the score.

In addition, in an embodiment, the first analysis function 351 executes the first analysis further based on the past medical information of the subject.

As a result, the lesion determination accuracy can be further improved.

In addition, in an embodiment, the second analysis function 352 executes the second analysis further based on the past medical information of the subject.

As a result, the lesion determination accuracy can be further improved.

In addition, in an embodiment, the determination function 353 determines the matching degree in consideration of the likelihood of the result of the first analysis and the likelihood of the result of the second analysis.

As a result, since the attribute of the minority can be reflected in the matching degree, the lesion determination accuracy can be further improved.

In addition, in an embodiment, in a case where the matching degree is equal to or greater than the first threshold and the first and second analysis results are benign, the analysis condition setting function 355 sets analysis conditions for reducing the false negative determination.

As a result, the determination can be appropriately performed according to the matching degree and the result of the second analysis.

In addition, in an embodiment, in a case where the matching degree is greater than the first threshold and the first and second analysis results are malignant, the analysis condition setting function 355 sets analysis conditions for reducing the false positive determination or detecting metastasis and recurrence of a lesion.

As a result, the determination can be appropriately performed according to the matching degree and the result of the second analysis.

In addition, in an embodiment, in a case where the matching degree is equal to or less than the first threshold and is equal to or greater than the second threshold, which is less than the first threshold, the analysis condition setting function 355 sets analysis conditions for reducing the false negative determination or detecting metastasis and recurrence of a lesion.

As a result, the determination can be appropriately performed according to the matching degree.

In addition, in an embodiment, in a case where the matching degree is less than the second threshold and the result of the first analysis is benign or absent, the analysis condition setting function 355 sets analysis conditions for early detection of a lesion.

As a result, the determination can be appropriately performed according to the matching degree and the result of the first analysis.

In addition, in an embodiment, in a case where the matching degree is less than the second threshold and the result of the first analysis is benign, the analysis condition setting function 355 sets analysis conditions for reducing the false positive determination based on an image or detecting metastasis and recurrence of a lesion.

As a result, the determination can be appropriately performed according to the matching degree and the result of the first analysis.

In addition, in an embodiment, in a case where the matching degree is less than the second threshold and the result of the first analysis is malignant, the analysis condition setting function 355 sets analysis conditions for reducing the false negative determination based on an image or detecting metastasis and recurrence of a lesion.

As a result, the determination can be appropriately performed according to the matching degree and the result of the first analysis.

Note that the processing circuitry in the embodiments described above process the medical image and the specimen examination result as the input data, but in another embodiment the processing circuitry may process input data other than them. For example, the processing circuitry may process Electric Medical Record (EMR) in addition to the medical image and the specimen examination result as the input data. In this case, the processing circuitry may determine the matching degree among the three kinds of the input data. As a result, diagnosis support information reflecting information which is not included in the medical image and the specimen examination result can be obtained.

Note that the term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)). The processor realizes the function by reading and executing the program stored in the storage circuit. Note that, instead of storing the program in the storage circuit, the program may be directly incorporated in the circuit of the processor. In this case, the processor realizes the function by reading and executing the program incorporated into the circuit. Note that the processor is not limited to a case of being configured as a single processor circuit, and a plurality of independent circuits may be combined to be configured as one processor to realize the function. Furthermore, a plurality of components in FIG. 2 may be integrated into one processor to realize the function.

According to at least one embodiment described above, the lesion determination accuracy can be improved.

Although several embodiments have been described above, these embodiments have been presented only as examples, and are not intended to limit the scope of the invention. The novel apparatuses and methods described in the present specification can be implemented in various other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

### Reference Signs List

What is claimed is:

1. A medical information processing apparatus comprising:
processing circuitry configured to
execute a first analysis based on a medical image of a subject,
execute a second analysis based on a specimen examination result of the subject,
execute determination of a matching degree between a result of the first analysis and a result of the second analysis, and
execute a third analysis based on the medical image and the specimen examination result and determine diagnosis support information based on the medical image and the specimen examination result under analysis conditions based on the determination result of the matching degree.

2. The medical information processing apparatus according to claim 1, wherein
the specimen examination result includes a value of a biomarker included in a specimen collected from the subject, and
the processing circuitry is further configured to
execute the second analysis based on the value of the biomarker,
execute the third analysis using machine learning,
set the analysis conditions by setting at least one of a parameter, a mode, and a network of the machine learning, and
execute the third analysis according to the set analysis conditions.

3. The medical information processing apparatus according to claim 2, wherein the parameter is a weight of an attribute of the biomarker contributing to the third analysis using the machine learning.

4. The medical information processing apparatus according to claim 3, wherein the processing circuitry is further configured to set the weight of the attribute of the biomarker contributing to the third analysis by determining a purpose of the third analysis based on the determination result of the matching degree, the first analysis result, and the second analysis result, determining an attribute of a biomarker to be noted based on the determined purpose of the third analysis, and weighting the determined attribute according to a degree of contribution of the determined attribute contributing to the second analysis.

5. The medical information processing apparatus according to claim 4, wherein the processing circuitry is further configured to, when the degree of contribution of the determined attribute contributing to the second analysis is low, weight the determined attribute more heavily than when the degree of contribution is high.

6. The medical information processing apparatus according to claim 4, wherein
the processing circuitry is further configured to
execute the second analysis using machine learning,
acquire the degree of contribution from a result of the machine learning used in the second analysis,
calculate a score according to the acquired degree of contribution, and
weight the determined attribute based on the calculated score.

7. The medical information processing apparatus according to claim 1, wherein the processing circuitry is further configured to execute the first analysis further based on the past medical information of the subject.

8. The medical information processing apparatus according to claim 1, wherein the processing circuitry is further configured to execute the second analysis further based on the past medical information of the subject.

9. The medical information processing apparatus according to claim 1, wherein the processing circuitry is further configured to determine the matching degree in consideration of likelihood of the first analysis result and likelihood of the second analysis result.

10. The medical information processing apparatus according to claim 2, wherein the processing circuitry is further configured to, in a case where the matching degree is greater than a first threshold and the first and second analysis results are benign, set the analysis conditions for reducing false negative determination.

11. The medical information processing apparatus according to claim 10, wherein the processing circuitry is further configured to, in a case where the matching degree is greater than the first threshold and the first and second analysis results are malignant, set the analysis conditions for reducing false positive determination or detecting metastasis and recurrence of a lesion.

12. The medical information processing apparatus according to claim 10, wherein the processing circuitry is further configured to, in a case where the matching degree is equal to or less than the first threshold and is equal to or greater than a second threshold, which is less than the first threshold, set the analysis conditions for reducing false negative determination or detecting metastasis and recurrence of a lesion.

13. The medical information processing apparatus according to claim 10, wherein the processing circuitry is further configured to, in a case where the matching degree is less than a second threshold, which is less than the first threshold, and the first analysis result is benign or absent, set the analysis conditions for early detection of a lesion.

14. The medical information processing apparatus according to claim 10, wherein the processing circuitry is further configured to, in a case where the matching degree is less than a second threshold, which is less than the first threshold, and the first analysis result is benign, set the analysis conditions for reducing false positive determination based on an image or detecting metastasis and recurrence of a lesion.

15. The medical information processing apparatus according to claim 10, wherein the processing circuitry is further configured to, in a case where the matching degree is less than a second threshold, which is less than the first threshold, and the first analysis results is malignant, set the analysis conditions for reducing false negative determination based on an image or detecting metastasis and recurrence of a lesion.

16. The medical information processing apparatus according to claim 4, wherein the attribute of the biomarker is use of the biomarker.

17. The medical information processing apparatus according to claim 1, wherein the specimen examination result includes values of a plurality of kinds of biomarkers, and the processing circuitry execute the second analysis based on the values of the plurality of kinds of biomarkers.

18. The medical information processing apparatus according to claim 16, wherein the attribute of the biomarker includes at least one of a general-purpose tumor marker, determination of triple negative breast cancer, determination of a subtype other than triple negative breast cancer, detection of metastasis, early detection, determination of fibroadenoma and determination of cancer recurrence.

19. A medical information processing method comprising:
executing a first analysis based on a medical image of a subject;
executing a second analysis based on a specimen examination result of the subject;
executing determination of a matching degree between a result of the first analysis and a result of the second analysis; and
executing a third analysis based on the medical image and the specimen examination result and determining diagnosis support information based on the medical image and the specimen examination result under analysis conditions based on the determination result of the matching degree.

20. A non-transitory computer-readable storage medium recording a program, the program causing a computer to execute:
a process of executing a first analysis based on a medical image of a subject;
a process of executing a second analysis based on a specimen examination result of the subject;
a process of executing determination of a matching degree between a result of the first analysis and a result of the second analysis; and
a process of executing a third analysis based on the medical image and the specimen examination result and determining diagnosis support information based on the medical image and the specimen examination result under analysis conditions based on the determination result of the matching degree.
